(19) United States
(12) Patent Application Publication
(10) Pub. No.: US 2005/0155609 A1
(43) Pub. Date: Jul. 21, 2005

(54) Adjustable binding-fixative-massaging device for condoms

(76) Inventor: Chin-Tien Lin, Keelung City (TW)

Correspondence Address: RABIN & CHAMPAGNE, P.C., Suite 500, 1101 14th Street, N.W., Washington, DC, 20005, US

(21) Appl. No.: 10/757,539

(22) Filed: Jan. 15, 2004

(51) Int. Cl. A61F 6/04

(52) U.S. Cl. 128/844

(57) ABSTRACT

This innovation relates to an adjustable binding-fixative-massaging device for condoms, referring to a fastener design adapting to the caliber of the root of the male penis for fine-adjustment binding-fixation of condoms enveloping penises, to prevent slipping off of condoms during coitus; with providing a massaging device of pipe-shaped body with one big and/or numerous small massaging protrusion/s and a vibration-transmitting device through a central hole of the pipe, for forming high-frequency oscillation to intensify erotic massage of the protrusions stimulating the female clitoris and increasing the efficacy of delight during coitus.

### BACKGROUND OF THE INVENTION

(a) Field of the Invention

This innovation relates to an adjustable binding-fixative-massaging device for condoms, especially referring to a fastener design adapting to the caliber of the male penis and a fastener strip for fine-adjustment binding-fixation, to achieve binding-fixing condoms enveloping penises and increasing the efficacy of delight during coitus.

(b) Description of the Prior Art

Erotic delight is a very important a behavior in marriage, for increasing the intimacy between the two genders. That is, erotic delight is not simply for propagation of progeny. Contraception during coitus takes an in-depth understanding into the physiological cycle of the coital partner, with engagement within the safe period of the female physiology. Only the safe period of the female is not absolutely safe, but there are still chances of impregnation. The reason for which is extremely complicated, thus the need for implementation for contraception, such as: intra-uterine contraceptive devices, tubal ligation, pills or condoms etc. Among which the most used and accepted should be condoms for men, for their ease and convenience. The usage is to envelope the erect penis during coitus to form a blocking type of contraception. But through the engagement, with the reciprocating movement of the male penis in the female vagina, condoms frequently slip off, constituting an embarrassment of the rough process.

Further during coitus, there is great difference between the orgasm of the two genders: according to questionnaire data of THE KINSEY INSTITUTE NEW REPORT ON SEX, about 10% of the women fail to achieve orgasm no matter what method is used; and 50 to 75% of women fail to achieve orgasm simply relying on the reciprocation of the penis. Thus there is a need for other techniques, such as: stimulation of the clitoris to massage to climax. So the medical field suggests the use of erotic massage prostheses to stimulate the sexual sensitive region.

Products having to do with erotic massage are variegated, to be generally categorized into rod-shaped massagers, vibratory ball massager or silicon rubber binding ring for massage 10. During coitus, use of binding rings for massage 10 is more prevalent, because it binds-fixes to the root of the male penis and during coitus stimulates the female clitoris with protrusions 101 of the massage part 100 on the upper edge of the binding ring for massage 10, additionally with the transmitted high-frequency from the vibration device 20 plugged on the massage part 100. Because the protrusion 101 is a relatively larger firm body, its resilience and softness are not excellent; the female clitoris under reciprocating thrusts of this less soft protrusion 101, experiences serious discomfort.

Although said binding ring for massage 10 massages/stimulates the female clitoris, it is a uni-diameter ring; even with tensile extensibility, it fails to adjust to penises of all sizes and effect a binding-fixation. So manufacturers have to use many molds of different sizes to produce binding rings for massage 10 of different sizes, to meet the demand of consumers with different sizes of penises, with retailers made to purchase a larger amount of binding rings for massage 10 of different sizes to facilitate the usage demand of customers. This, would hardly meet the effective economic management principle of the manufacturers and marketers.

In light of this shortcoming of the conventional structure, the innovator of this application gathered his ample experience accumulated through many years engaged in the product research and production of condoms and its like, actively researched and experimented, and finally successfully accomplished the development of an adjustable binding-fixative massage device for condoms, to effectively solve the shortcoming of conventional structures, and achieve prevention of slipping off of condoms during coitus, also increasing stimulation-massage to the sensitive region of females, further achieving easy production, convenient use and effectively raising the quality and function of the product.

As to the structure, objective, manner and spirit of this innovation, the Patent Office is invited to refer to the following illustrations and descriptions to completely understand this innovation.

### SUMMARY OF THE INVENTION

This innovation relates to an adjustable binding-fixative-massaging device for condoms, especially referring to a fastener design adapting to the caliber of the root of the male penis for fine-adjustment binding-fixation, to achieve binding-fixing condoms enveloping penises, to prevent slipping off of condoms enveloping penises in reciprocal movements in the vagina during coitus; with providing a massaging device of pipe-shaped body with numerous massaging protrusions and a vibration-transmitting device at a central hole of the pipe, for forming high-frequency oscillation to intensify erotic massage of the protrusions stimulating the female clitoris and increasing the efficacy of delight during coitus.

### BRIEF DESCRIPTION OF DRAWINGS

FIG. 1A and FIG. 1B are the drawings of the brief presentation and usage state of the conventional massage binding ring, respectively.

FIG. 2 is the 3D drawing of this innovation.

FIG. 3 is the drawing of the perspective view of the oscillation shut off of the vibration-transmitting device of this innovation.

FIG. 4. is the drawing of the perspective view of the oscillation activation of the vibration-transmitting device of this innovation.

FIGS. 5A and 5B are the drawings of the 3D drawing of the spinning knob and bulging hole at one end of the vibration-transmitting device of this innovation, repectively.

FIG. 6 is the drawing of the state of Implementation of this innovation.

FIG. 7 is another drawing of the state of Implementation of this innovation.

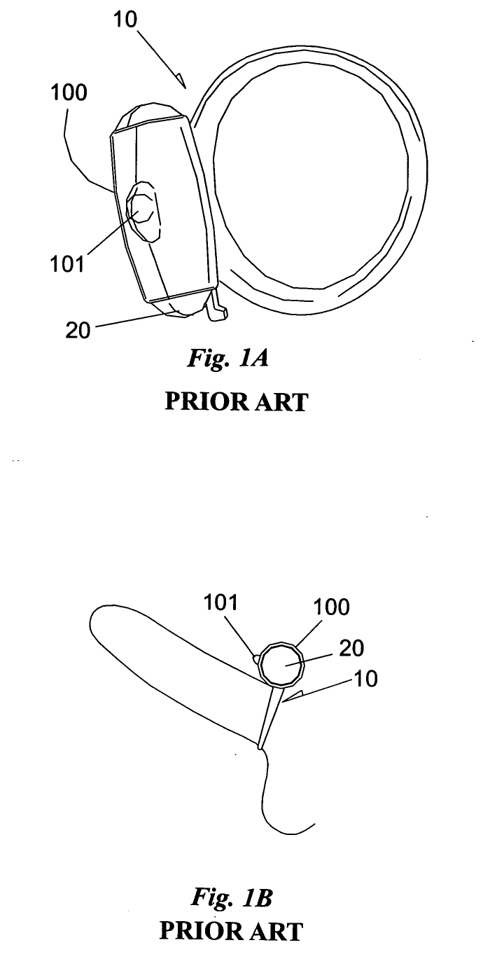

### BRIEF DESCRIPTION OF NUMERALS

10 Massage binding ring
100 Massage part
101 Protrusion
20 Vibration device
50 Binding-fixative-massaging device
501 Pipe-shaped body
502 Massaging protrusions
503 Fastener strip
503a Fastener beads
504 Fastener bead blocker
504a Fastener hole
60 Vibration-transmitting device
60a Bulging hole
601 Spinning knob
601a Spin rod
601b Bulging pin
601c Bulging pin
61 Spring strip
62 Contact strip
63 Movable conducting strip
70 Transmitting body
80 Battery

### DETAILED DESCRIPTION OF PREFERRED EMBODIMENT

This innovation is an adjustable binding-fixative massage device, referring to a fastener design to adapt to the caliber of the root of the male penis with fine-adjusting binding-fixation, to achieve binding-fixation of the condom enveloped on the penis, to prevent the enveloping condom from slipping off due to reciprocating movement of the penis in the vagina during coitus; with a massage device of pipe-shaped body covered with numerous massaging protrusions, a central hole of the pipe where a vibration-transmitting device is provided to generate high-frequency oscillations for intensified erotic massage, stimulating the female clitoris with a massaging protrusion and increasing the efficacy of erotic delight.

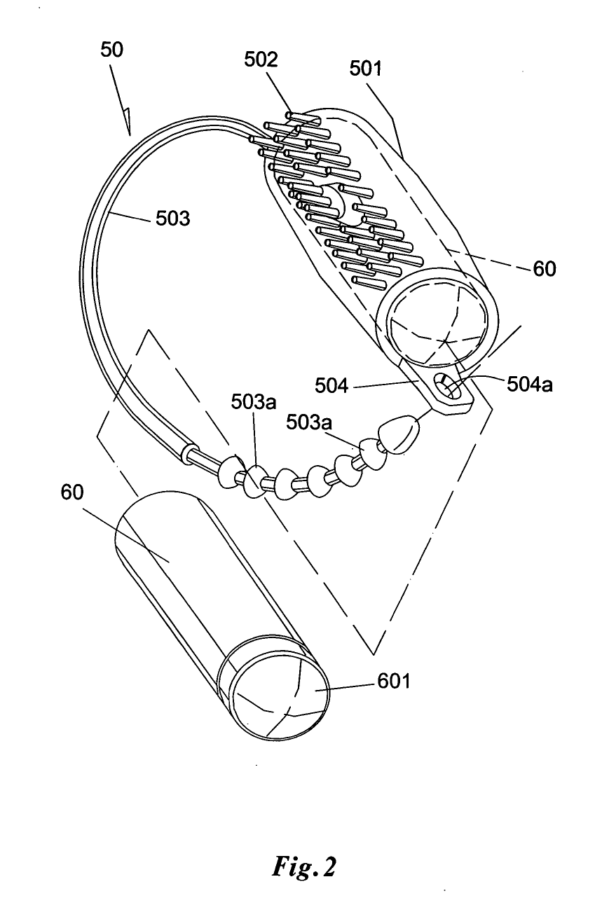

The innovation is an adjustable binding-fixative device for condoms, (please refer to FIG. 2), a massage device 50 with integral soft resilient material, which comprising a pipe-shaped body 501 and a vibration-transmitting device 60, of which the center of the surface of the pipe-shaped body 501 is provided with a proper protrusion with extension of several soft protrusions 502, and around said protrusion and on both of the sides plural soft massage protrusions 502 are provided (to thrust the female clitoris during coitus), said pipe-shaped body 501 is provided with a vibration-transmitting device 60 at the center hole, and with a fastener strip 503 and a fastener bead blocker 504 extended from both ends of the pipe-shaped body 501, the center of the fastener bead blocker 504 is provided with a fastener hole 504a, and one end of the fastener strip 503 is provided with several semi-spherical fastener beads 503a stringed with spaces in between, and the tip of the fastener strip 503 is provided with a cone-shaped fastener bead 503a to insert into the fastener hole 504a of the fastener body 504; further, (please refer to FIG. 3,4 and 5), with the vibration-transmitting device 60 mainly provided with a spring strip 61, of which one end is shaped into a bent contact strip 62 to be in contact with the outer ridge of the transmitting body 70, while the other end is bent into a movable conducting strip 63 at the rear of the battery 80 of the transmitting body, and on the other side of the vibration-transmitting device a bulging hole 60a is provided for the spin rod 601a of the spin knob 601 to insert and spin, and provided on different places on said spin rod 601a there are bulging pins 601b and 601c.

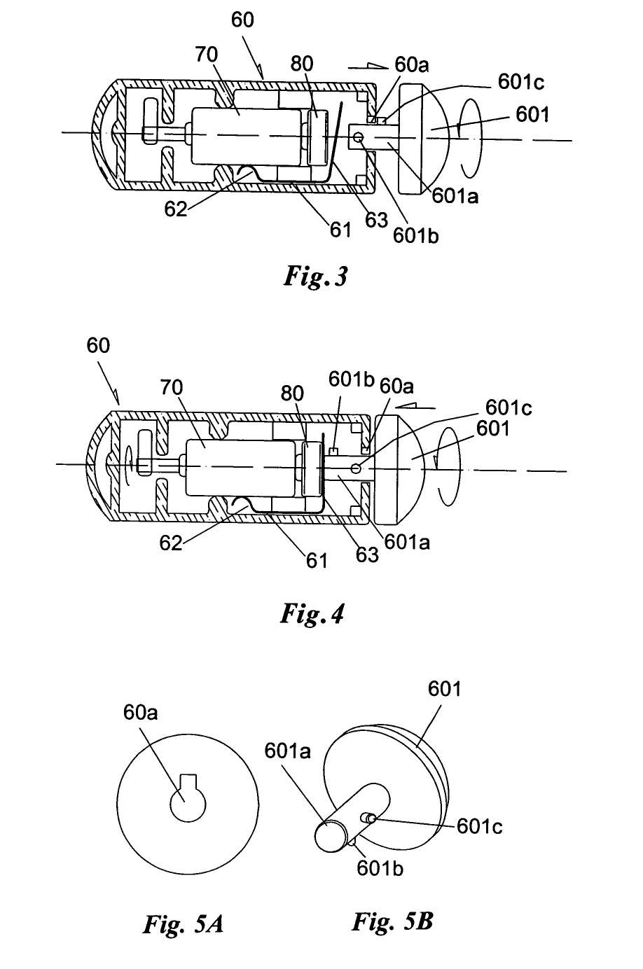

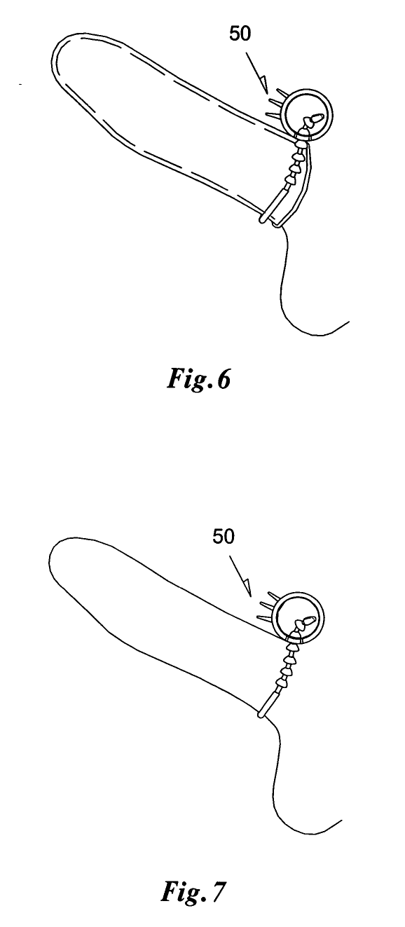

Through, (please refer to FIG. 2, 6 and 7), inserting the cone-shaped fastener bead 503a at the end of fastener strip 503 of one end of the pipe-shaped body 501 of the binding-fixative-massaging device 50 into the fastener hole 504a of the fastener bead blocker 504 at the other end of the pipe-shaped body 501 to adjust the fastener strip 503 to the proper tightness for a penis with the semi-spherical fastener beads 503a to butt to the fastener bead blocker 504 without slipping off, also to fix to the root of the penis the condom; during the engagement of coitus to spin the spinning knob 601 of the vibration-transmitting device 60 (please refer to FIG. 4, FIG. 5A and FIG. 5B) and make spin rod 601a completely insert into bulging hole 60a to push the spring strip 61 of the movable conducting strip 63 for contacting the battery 80 and conduct-activate the transmitting body 70 to spin and generate oscillations and also to drive the massaging protrusions 502 on the surface of the pipe-shaped body 501; and if spinning the spinning knob 601 further, making the bulging pin 601c of the spin rod 601a butt to the inner wall of the shell, to make the battery continuously activate the spin-vibration; and to stop oscillating the massage (please refer to FIG. 3) by spinning the spinning knob 601 for the bulging pin 601c of the spin rod 601a to be withdrawn through bulging hole 60a for the other bulging pin 601b of the spin rod 601a to butt to the inner wall of the shell and prevent the spinning knob 601 from slipping and the movable conducting strip 63 of the spring strip 61 to be detached from the battery 80, then the transmitting body 70 would stop spinning and vibrating.

1. An adjustable binding-fixative-massaging device for condoms—an integral binding-fixative-massaging device from soft and resilient material, comprises a pipe-shaped body and a vibration-transmitting device to be fixed/installed inside which, at the center of the pipe-shaped body a proper protrusion and several soft protrusions in extension are provided, further around and by two sides of said protrusion being provided plural soft massage protrusions, and at the central hole of the pipe-shaped body there is a vibration-transmitting device; and of the said pipe-shaped body one end being provided with a fastener strip in extension and the other end a fastener bead blocker with a fastener hole at its center, in which near the end of said fastener strip there are several semi-spherical fastener beads stringed together with spaces in between and at this end of the fastener strip is provided one cone-shaped fastener bead for inserting into the fastener hole of the fastener bead blocker; further a vibration-transmitting device, mainly a spring strip with one end bent into a contact strip for butting to the outer edge of the transmitting body and the other end bent upward to form a movable conducting strip behind the battery of the transmitting body, still one end of the vibration-transmitting device is provided with a bulging hole for a spin rod with spinning knob and bulging pins on different sites on said spin rod, to be inserted and spun;
through the above structures to pierce/butt the fastener strip at one end of the pipe-shaped body into the fastener hole of the fastener bead blocker at the other end of the pipe-shaped body to adjust a proper binding tightness and bind/fix the whole binding-fixative-massaging device at the root of a penis, and make or break with the spinning knob of the vibration-transmitting device to drive the movable conducting strip of the inner spring strip into or out of contact with the battery, to start or shut off the vibration-transmitting device.
